(19) United States
(12) Patent Application Publication
(10) Pub. No.: US 2021/0300990 A1
(43) Pub. Date: Sep. 30, 2021

(54) METHOD FOR THE PRODUCTION OF BI-FUNCTIONAL CELLS TO TREAT NEOPLASIA

(71) Applicant: Rigenerand S.R.L., Medolla (IT)

(72) Inventors: Giulia Golinelli, Vignola (IT); Giulia Grisendi, Reggio Emilia (IT); Carlotta Spano, Modena (IT); Olivia Candini, Crevalcore (IT); Massimo Dominici, Ferrara (IT)

(21) Appl. No.: 17/263,953

(22) PCT Filed: Jul. 30, 2019
(86) PCT No.: PCT/IT2019/050177

§ 371 Date: Jan. 28, 2021

(30) Foreign Application Priority Data
Jul. 30, 2018 (IT) 102018000007601

(51) Int. Cl. C07K 14/705; C07K 16/30; C12N 15/86; C12N 5/077; C12N 5/0775

(52) U.S. Cl. CPC C07K 14/70575; C07K 16/3084; C12N 15/86; C12N 5/0652; C12N 5/0667; C07K 2317/622; C07K 2317/73; C07K 2319/33; C07K 2317/76; C12N 2740/15043; C12N 2740/10043; C12N 2510/00; C07K 2319/30

(57) ABSTRACT

Method for the production of bi-functional cells comprising engineering a starting cell population with a phenotype attributable to human pericytes extracted from adipose tissue (AD-PC), obtaining engineered cells, known as bi-functional AD-PCs, expressing (which means that they produce) simultaneously both the anti-tumor molecule TRAIL and also the truncated form of a chimeric receptor targeted against the GD2 antigen (GD2 tCAR); this dual targeting (understood as reaching a specific target), based on affinity and mediated by both TRAIL and also GD2 tCAR, supports the prediction of combining site-specificity with a prolonged retention of AD-PCs in tumors expressing the antigen GD2, so as to achieve a more effective release of TRAIL for still incurable tumors.

### FIELD OF THE INVENTION

The present invention concerns a method for the production of bi-functional AD-PCs, co-expressing TRAIL protein and the GD2 tCAR receptor, so as to re-direct the AD-PCs that release the anti-tumor protein TRAIL against tumors expressing the GD2 antigen.

### BACKGROUND OF THE INVENTION

Adult progenitor cells, including a cell population with a phenotype attributable to human pericytes (hereafter PCs), are being studied as cell vehicles for the release of anti-tumor molecules.

PCs can be obtained from different sources and possess an innate tropism for tumors, as presumed precursors of tumor stromal cells.

PCs can be easily isolated and engineered (that is, genetically modified) with the use of viral vectors.

The direct targeting of primary tumors and the respective metastases with PCs engineered to produce anti-tumor molecules currently represents a promising cell-based strategy, in particular for compounds equipped with a reduced half-life or scarce bio-availability in target sites.

PCs have the potential advantages of offering a prolonged and concentrated release of therapeutic molecules, reducing non-selective targeting, thus representing an encouraging approach to improve the effectiveness of standard therapeutic treatments.

The ability of PCs for “homing”, that is, to locate themselves in correspondence with the tumor site, has been demonstrated for different types of cancer, including glioma, breast cancer, sarcoma, colon cancer, ovarian cancer, carcinomas of the pancreas and carcinomas of the lung.

It is known that the pro-apoptotic agent Tumor necrosis factor-Related Apoptosis-Inducing Ligand (hereafter TRAIL in short), can be effectively released by PCs at tumor sites, acting on functional TRAIL receptor agonist (specifically DR4 and DR5), which are widely and preferably expressed on tumor cells.

From European patent EP2424979 a method for the production of medicaments to treat tumors is known which comprises preparing a retroviral vector that encodes a soluble TRAIL molecule (sTRAIL) and stably infects adipose pericytes (AD-PCs).

PCs that produce TRAIL have demonstrated cytotoxic activity in vitro and in vivo with respect to some tumors and are therefore particularly interesting in the context of cancer treatments.

However, this state of the art is affected by some problems.

A first problem is that, despite the tropism of PCs for the tumor microenvironment, it has been shown that the number of PCs in the tumor decreases over time and, for the complete therapy, different administrations of these cells may be required.

Therefore, particular attention is paid to methods for increasing PC affinity with target sites, so as to obtain better retention in tumor sites and a prolonged therapeutic effect therein.

Most of the preliminary work for targeting approaches based on binding affinity has been consolidated in the field of gene therapy in adoptive immunotherapy.

The maximum affinity has been obtained using immune molecules, such as antibodies, the T lymphocyte receptor (TCR), or their derivatives, such as the chimeric antigen receptors (hereafter CAR in short).

This type of targeting benefits from the presence of surface molecules uniquely or highly expressed by cancer cells.

A further problem is that the use of vehicle cells often performs a protective function on the therapeutic molecule conveyed but does not necessarily improve its release specificity which is critical to limit the off-target toxicity (that is, the so-called side effects).

In order to solve this problem, it has been found that affinity-based release by means of engineered vehicle cells increases the concentration of the agent on site and, indirectly, reduces its sequestration, dilution or inactivation in non-target tissues, thus further improving its efficacy.

Overall, the action of conveying a therapeutic agent greatly reduces the total dose required for therapeutic treatment and limits its toxicity.

Because PCs have intrinsic homing toward the tumor, affinity-based targeting strategies can further improve therapeutic release by PCs.

In detail, many neoplasia of neuroectodermal origin, including glioblastoma (GBM), lung microcytoma, melanoma, neuroblastoma and bone and soft tissue sarcomas, for example Ewing's sarcoma (ES), commonly express high levels of disialoganglioside GD2.

Furthermore, some breast cancer variants have been positive for disialoganglioside.

GD2 is a surface molecule with a restricted expression in normal tissues, thus representing a putative target for new therapeutic approaches.

Recently, two different GD2 targeting approaches have been developed, that is, with monoclonal antibodies targeted against GD2, and with immunotherapy with T-CAR lymphocytes.

The affinity domain of the CAR, represented by the single-chain fragment variable (scFv) deriving from the immunoglobulin targeted toward the tumor marker of interest, re-directs the cytotoxic function of the immune cells toward the specific target, thus increasing its localization in the tumor site.

Affinity-based cell targeting has also recently been applied to PCs.

Tumor-specific targeting and retention at the tumor site have been obtained, according to literature, by means of genetic modification of PCs with artificial receptors targeted against erbB2 in ovarian cancer and against EGFRvIII in GBM.

If on the one hand the interaction mechanism between TRAIL, released by PCs, and its receptors is known, on the contrary the way in which PCs can selectively localize and achieve targeting of the tumor is not known.

The invention focuses on optimizing the localization and targeting capacity of TRAIL producing PCs (PC TRAIL) in tumor sites.

Therefore, in order to maximize the therapeutic profile of PC TRAIL and minimize the side effects, a new strategy has been developed in which TRAIL is released by bi-functional PCs, which have been simultaneously engineered with TRAIL and with a truncated form, that is, missing the intracell signal transduction domain, of an anti GD2 CAR (GD2 tCAR).

By means of this immunoselective targeting, we aimed to direct these bi-functional PCs specifically against GD2 positive tumors.

The GD2 tCAR receptor as well as TRAIL were expressed in the PCs by means of infection with viral vectors.

Bi-functional PCs express high levels of both TRAIL and also GD2 tCAR preserving a robust anti-tumor activity against both GBM and also ES cell lines.

It is important to note that the anti-tumor action has therefore been further strengthened in bi-functional PCs, where the presence of GD2 tCAR improves the ability to establish cell-to-cell interactions thus allowing optimal targeting of GD2 expressing tumors.

Although the expression of GD2 tCAR does not act on the homing of PCs, it has been hypothesized that reinforcing the binding of PCs with tumor cells can increase PC retention in the tumor site, which in turn affects both the specificity and also the efficacy of the cell therapy approach.

According to the invention, the innate tropism of PCs for tumors coupled with the specific targeting mediated by the GD2 antigen could be advantageous in general to improve the therapeutic release of the TRAIL molecule for the treatment of GD2 positive malign tumors.

### OBJECTS OF THE INVENTION

One object of the present invention is to improve the state of the art.

Another object of the invention is to provide a method to engineer human PCs isolated from adipose tissue (AD-PCs) simultaneously both with TRAIL and also with the GD2 binding GD2 tCAR receptor, as a new powerful tool to re-direct the PCs producing the pro-apoptotic agent TRAIL against tumors expressing the GD2 antigen, for example against GBM and ES. The invasive nature of these tumors is the main cause of the failure of surgical resection and of standard therapeutic approaches.

Another object of the invention is to provide a method to produce engineered PCs with a marked tropism for the tumor as a possible therapeutic alternative, where these cells serve as a vehicle for anti-tumor substances with the aim of genetically modifying elements of the tumor microenvironment, in order to destroy the tumor from the inside, the so-called “Trojan horse” effect.

It has in fact been found that PCs persist inside the tumor stroma, settling in proximity to tumor cells and new vessels.

Current studies are directed toward optimizing both the specificity and also the effectiveness of PC-based cell therapy, in order to prolong the retention of PCs in the tumor and in parallel target a specific cell population.

According to one aspect, the invention provides a method to produce bi-functional PCs, engineered simultaneously with both TRAIL and GD2 tCAR, able to maintain the TRAIL-mediated cytotoxic capacity (that is, linked to the release of TRAIL by PCs), while at the same time acquiring a significant binding specificity to GD2-positive tumor cells.

As a confirmation of the purpose reached, in vitro data have shown that the simultaneous expression of the pro-apoptotic ligand TRAIL and of the GD2 tCAR protein does not influence the mortality produced on the tumor by the engineered PCs.

In parallel with the cytotoxic potential, the new properties acquired by the PCs engineered with GD2 tCAR were analyzed, and in particular the ability to bind GD2-expressing tumor cells, in a cell-to-cell interaction assay.

The number of interactions between PCs and tumor cells proved to be proportional to both the density of GD2 on the surface of tumor cells and also to the presence of GD2 tCAR on PCs.

It should be noted that the PCs engineered with the GD2 tCAR receptor have shown to bind more efficiently and specifically GD2-expressing tumor cells.

The cell-to-cell interaction assay has further demonstrated how the increased binding capacity of bi-functional PCs results in a more effective cytotoxic capacity against GD2-positive GBM cells, compared to PCs that express only TRAIL.

These data suggest that the expression of GD2 tCAR in bi-functional PCs allows specific targeting of tumor cells expressing GD2, at the same time enhancing the cytotoxic anti-tumor effect.

According to a further object of the invention, a first demonstration has been provided of a genetic modification of PCs simultaneously both with TRAIL and also with a chimeric receptor against an antigen expressed on the surface of tumor cells.

In detail, the bi-functional PCs comprise a nucleus, a cytoplasm, a stable modification which comprises simultaneously a TRAIL molecule and a GD2 tCAR molecule encoded by at least one viral vector and in which said viral vectors comprise retroviruses, and in particular lentiviruses.

These and other objects are achieved by a method to produce bi-functional cells, expressing both a molecule with anti-tumor activity and also a fraction of a monoclonal antibody targeted against a tumor antigen, according to the characteristics of claim 1.

Further characteristics are indicated in the dependent claims.

### BRIEF DESCRIPTION OF THE DRAWINGS

Further characteristics and advantages of the invention will become more evident from the description of an embodiment of a method to produce bi-functional PCs expressing both TRAIL and also GD2 tCAR, indicated below by way of a non-limiting example wherein:

FIG. 1 is an image showing the approaches based on bi-functional PCs.

FIG. 1a is the targeted killing with bi-functional PCs (indicated with 1) co-expressing GD2 tCAR (2) and a soluble trimeric TRAIL variant (sTRAIL; 3), therefore called bi-functional PC sTRAIL (1).

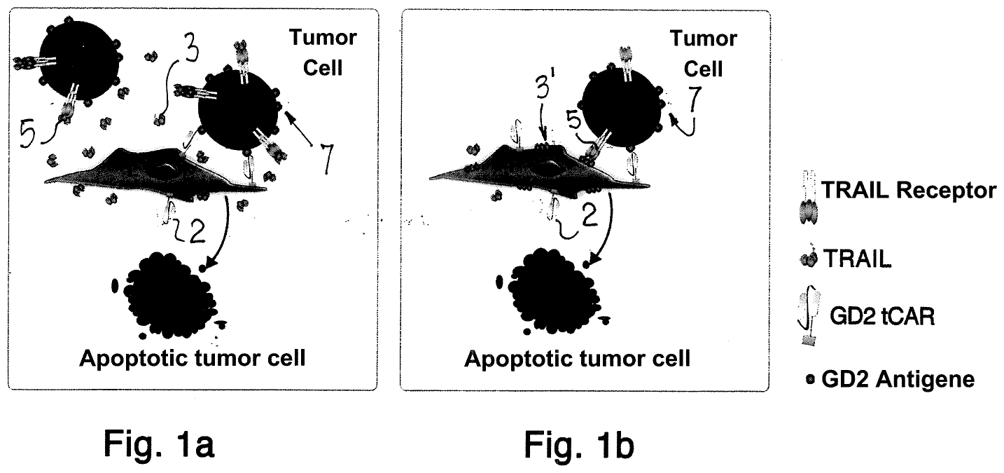

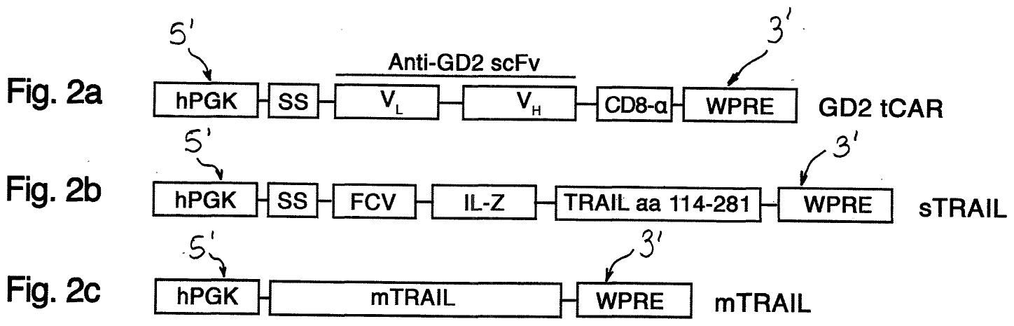

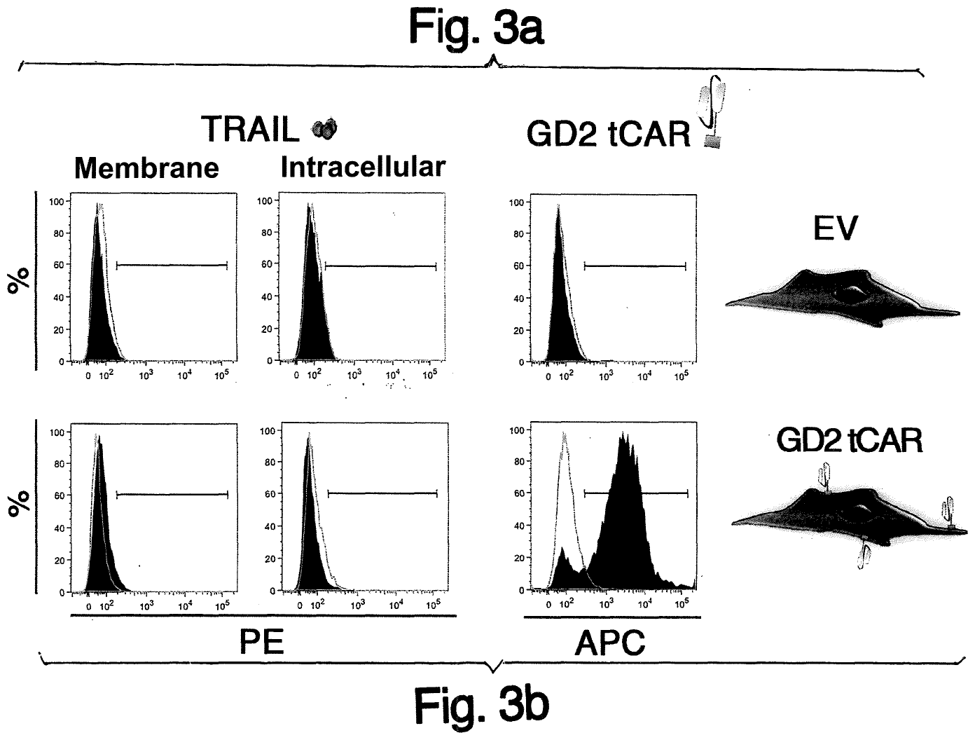

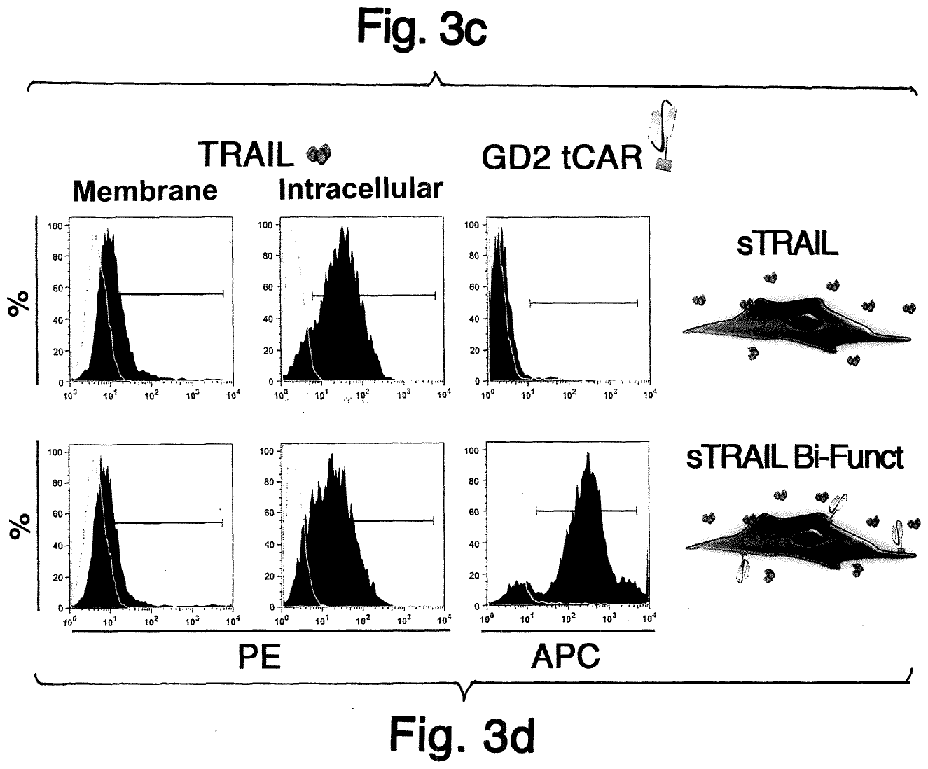

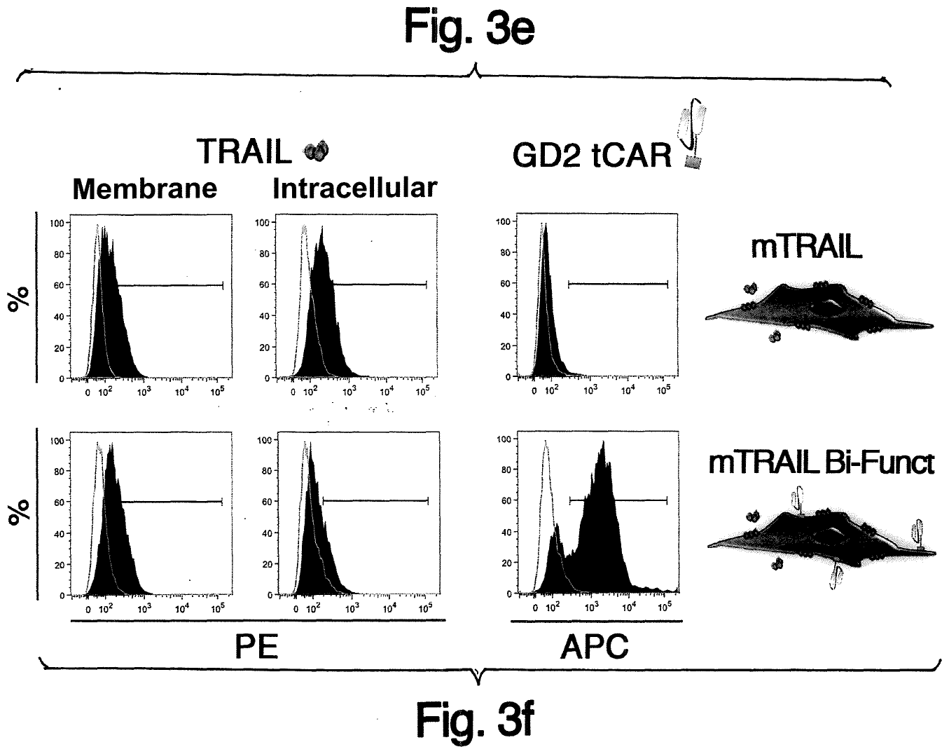

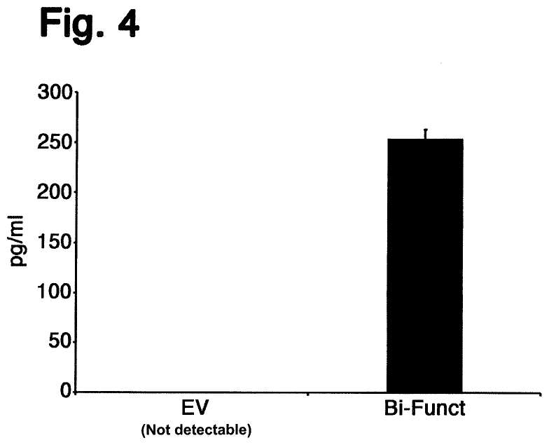

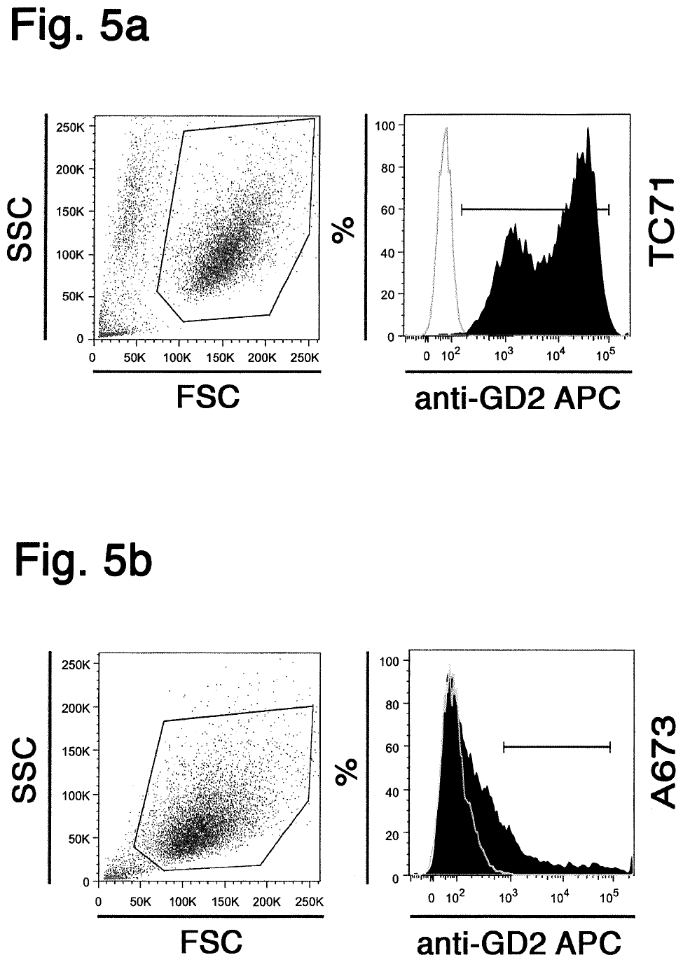

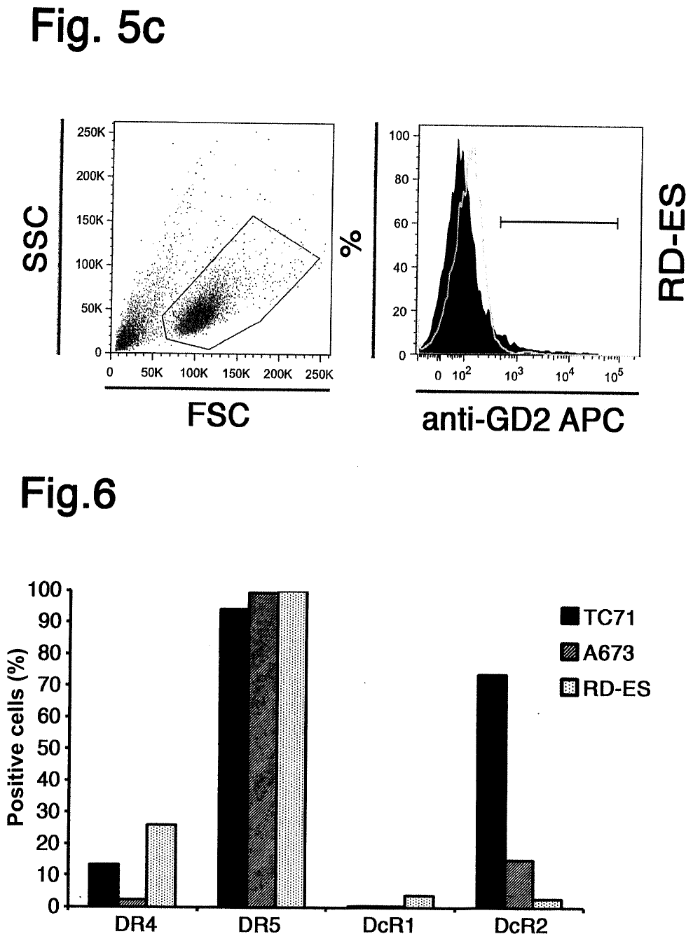

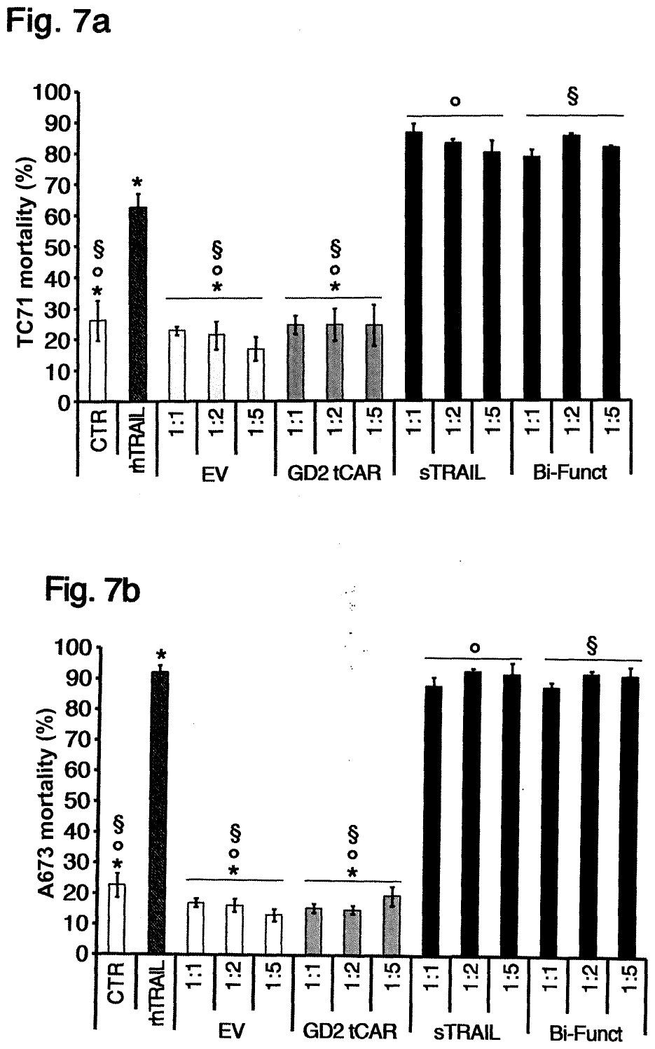

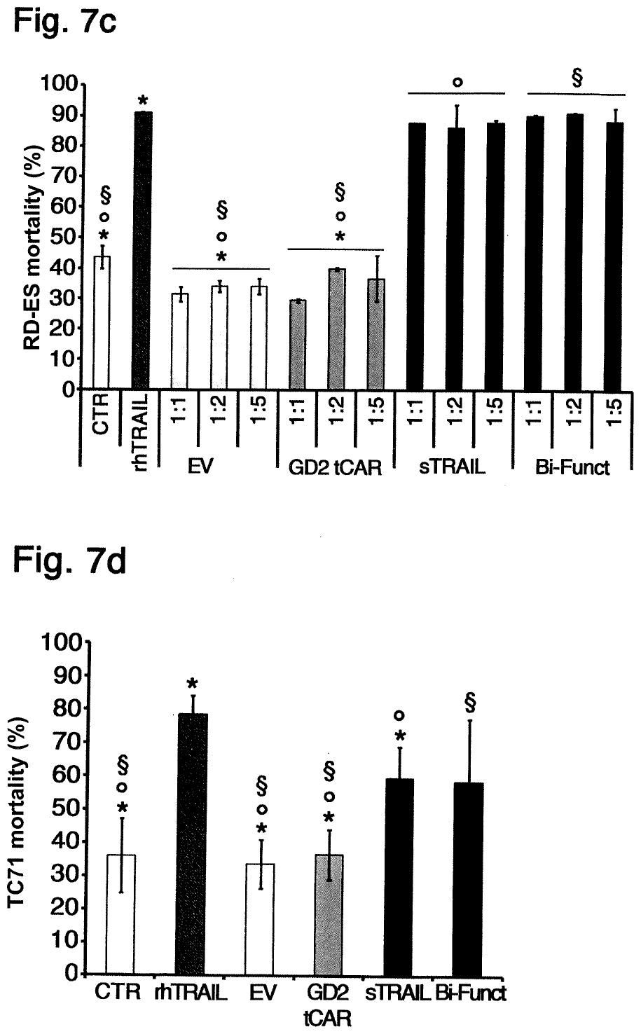

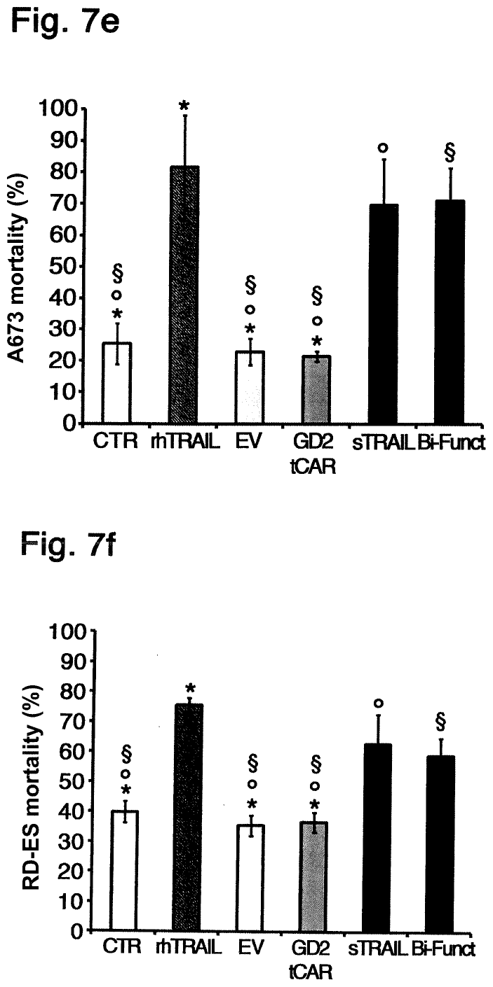

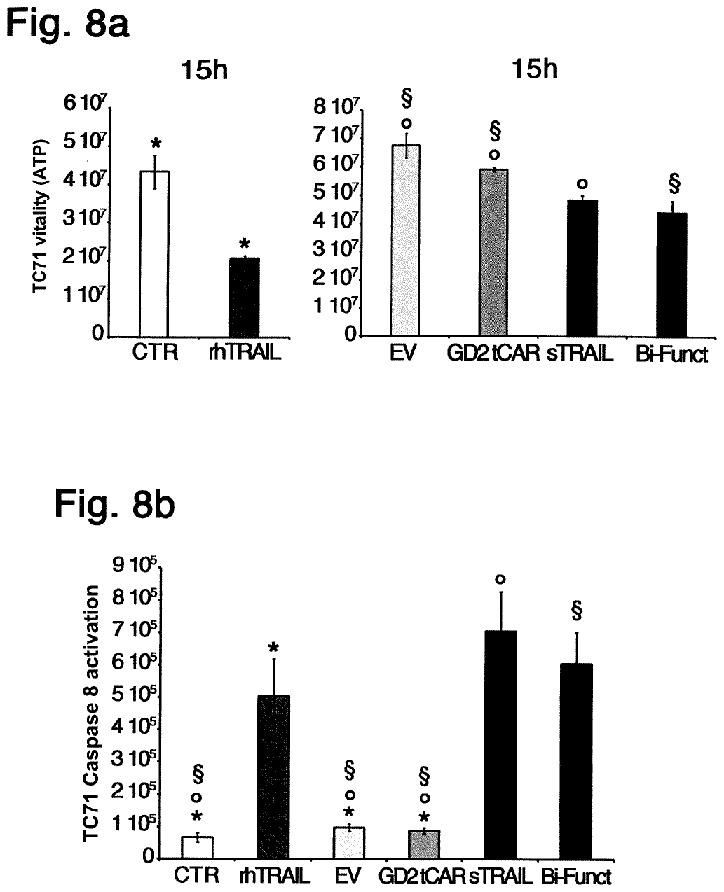

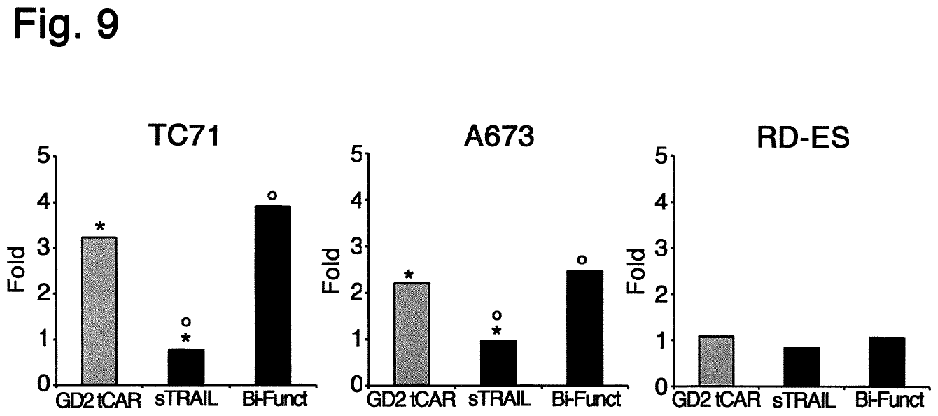

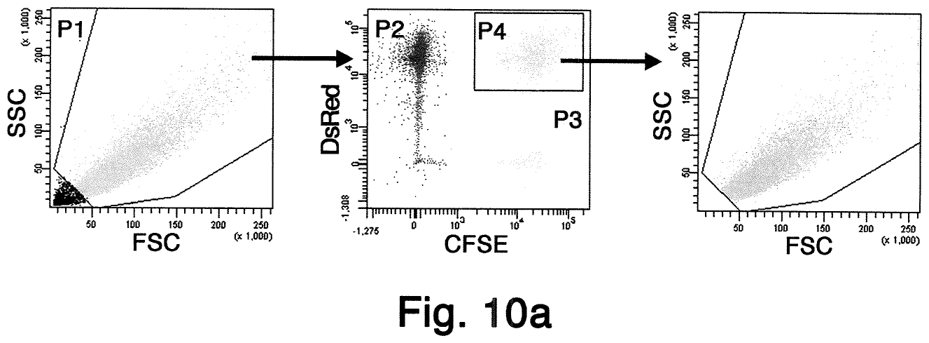

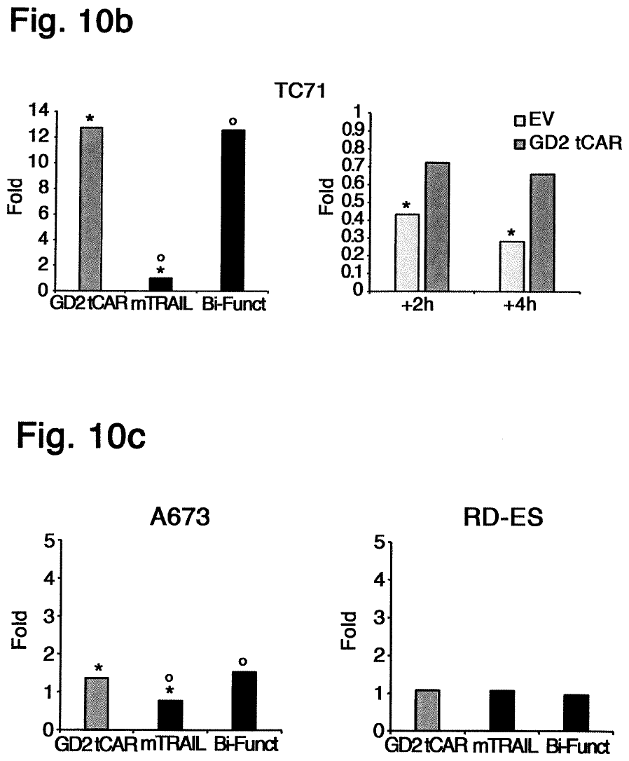

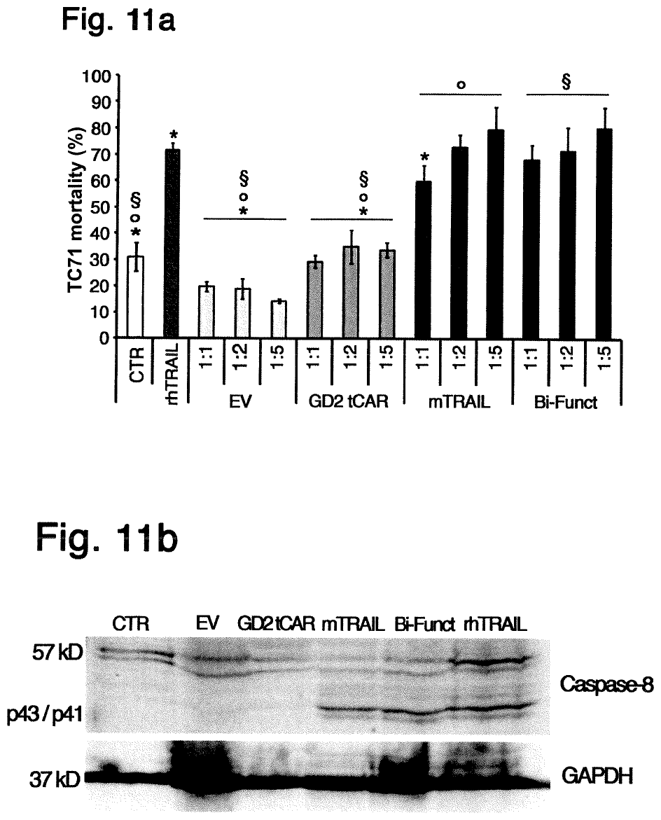

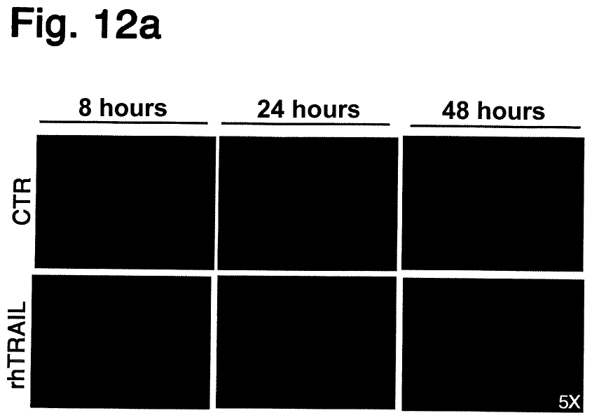

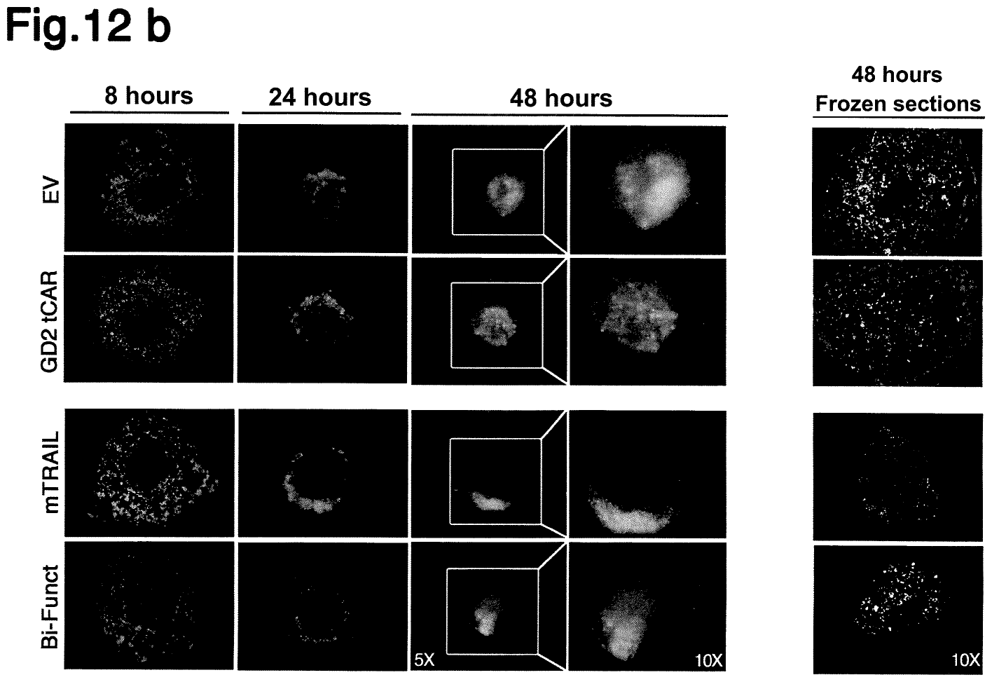

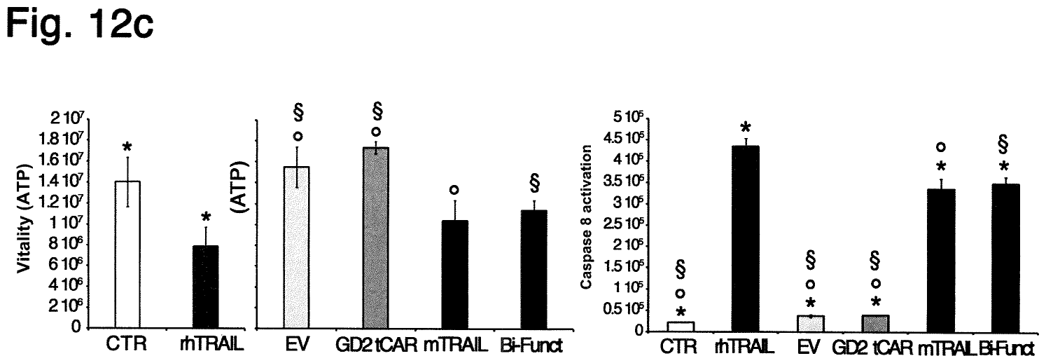

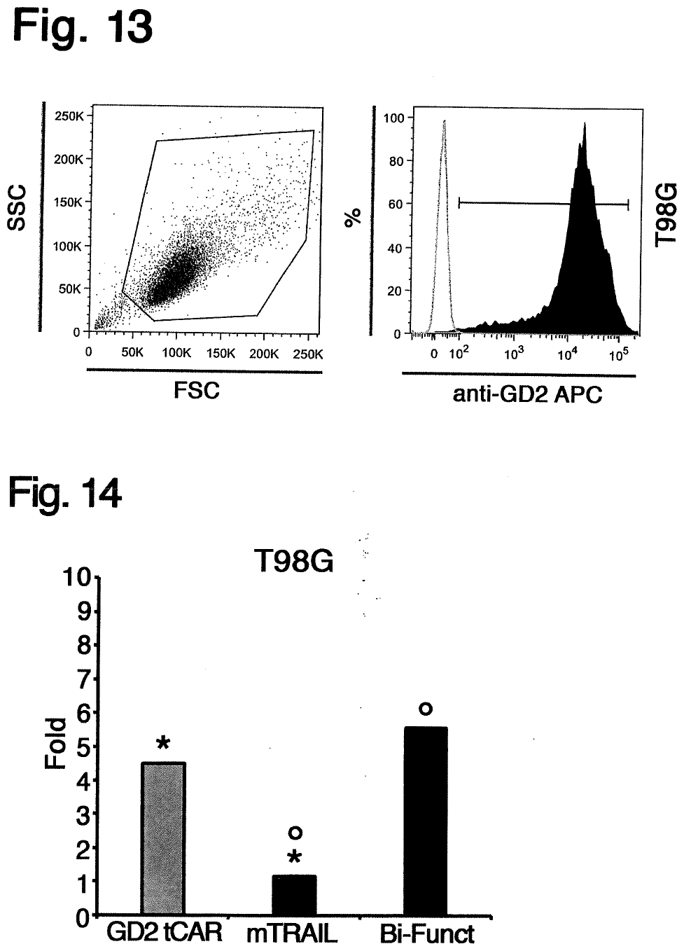

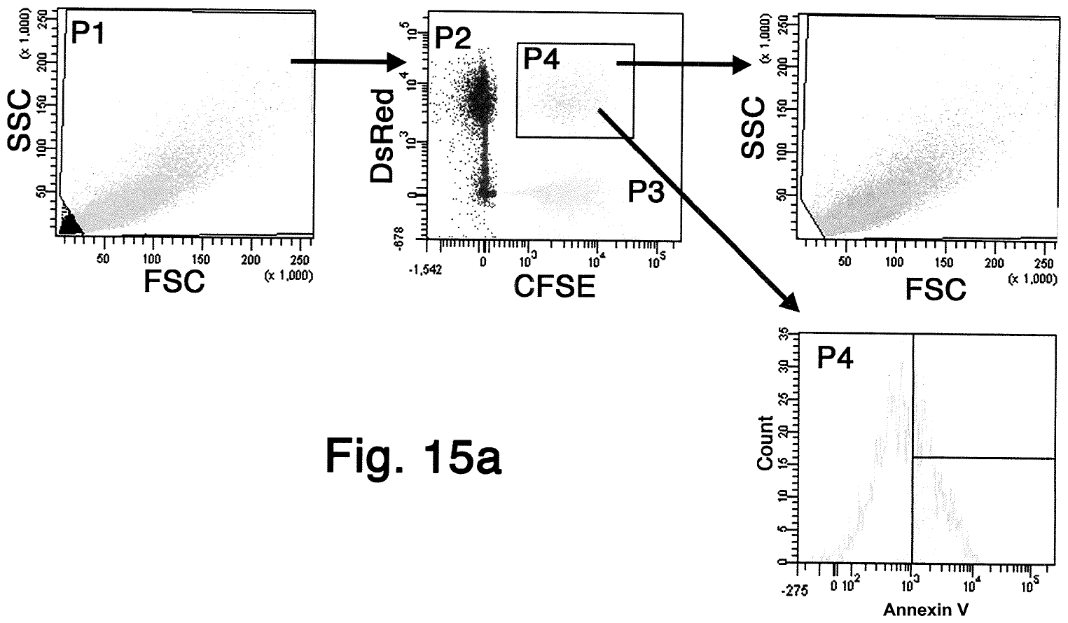

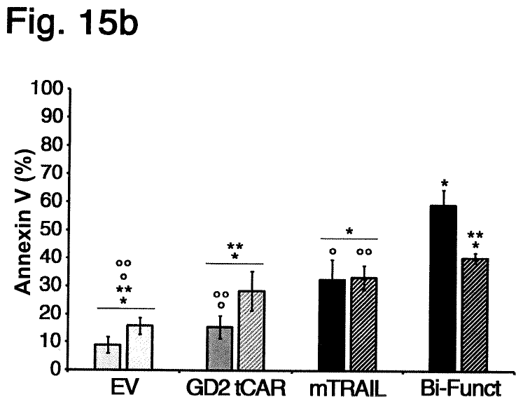

In FIG. 1a, it can be noted that sTRAIL (3) released from bi-functional PC sTRAIL (1), exerts its cytotoxic effect without the need for direct contact of the PCs with tumor cells (4), binding to a corresponding receptor (5) on the tumor cell and activating cell death by apoptosis (indicated with an apoptotic tumor cell, 6).

Since metastatic disease represents a major challenge for cancer patients, technology based on bi-functional PC sTRAIL is introduced with the aim of improving cell-to-cell interactions with the tumor and reaching distant metastatic sites by means of the release of sTRAIL.

According to the invention, the cytotoxic potential of bi-functional PC sTRAIL combined with targeting mediated by GD2 tCAR, can represent a new effective approach for the treatment of cancer even after metastatic dissemination.

FIG. 1b shows the targeted killing with bi-functional PCs (1′) expressing GD2 tCAR (2) together with a form of membrane-bound TRAIL (mTRAIL; indicated with 3′), hereafter called bi-functional PC mTRAIL (1′).

With this strategy, the tumor cell (4) is sent into apoptosis (indicated with the apoptotic tumor cell, 6) by means of direct cell-to-cell contact and consequent binding between mTRAIL (3′) on PCs and the respective receptor (5) on the tumor cell.

As can be seen in FIGS. 1a and 1b, the functionalization (that is, the introduction of an element that confers a function) of the surface of the bifunctional PCs (1 and 1′) with GD2 tCAR (2) increases the interaction capacity of the PCs with tumor cells (4) expressing a GD2 antigen (7), allowing the recognition of a selective cell target, with the ultimate object of increasing PC retention within the tumor site, which in turn affects both specificity and also the efficacy of cell-mediated administration of TRAIL.

FIG. 2a is the schematic representation of the construct expressing GD2 tCAR, in which the anti-GD2 scFv derived from an immunoglobulin M (IgM) has been fused with the transmembrane hinge domain of human CD8-α.

FIG. 2b shows the construct expressing sTRAIL which contains a secretion signal (SS) deriving from an immunoglobulin linked to a furin cleavage site (FCV) and to an isoleucine zipper (IL-Z) (which allows the formation of a trimeric structure) that is conjugated to the receptor binding domain of TRAIL (amino acids 114-281).

In FIG. 2c, it can be noted that the construct expressing mTRAIL carries the entire TRAIL human gene.

All the expression constructs described above are inserted between the lentiviral regulatory elements PGK and WPRE of the pCCL PGK WPRE vector.

Figs. from 3a to 3f show the expression of TRAIL variants (in detail mTRAIL and sTRAIL), both as a protein present at the cell membrane level and also at the intracell level (in the cytoplasm), and of the GD2 tCAR molecule on the cell surface verified by FACS (Fluorescent-Activated Cell Sorter) analysis of the engineered PCs.

Co-expression in PCs of GD2 tCAR together with mTRAIL or sTRAIL to produce respectively bi-functional PC mTRAIL and bi-functional PC sTRAIL (in the following drawings referred to as Bi-Funct) was obtained by infection with at least one retroviral vector, in particular lentiviral, although the optimal engineering method we apply provides to use different retroviral vectors (in particular lentivirals), represented in FIG. 2.

In the histograms specified below, the dark gray curve indicates the expression of TRAIL or of GD2 tCAR in PCs evaluated by labeling with specific antibodies while the light gray line represents the control produced by labeling with an antibody of identical isotype class but not targeted against TRAIL or GD2 tCAR. Specifically, the presence of TRAIL was evaluated with murine antibody conjugated to fluorochrome PE targeted against human TRAIL (produced by BioLegend, San Diego, Calif., US).

Intracell labeling was performed with Cytofix/Cytoperm kit (BD, Franklin Lakes, N.J., USA).

In order to detect GD2 tCAR, the PCs were incubated with anti-idiotype mouse sera containing antibodies able to recognize GD2 tCAR and subsequently with a secondary antibody conjugated to goat-produced fluorochrome APC and targeted against murine antibodies (BD).

FIG. 3a shows the analysis of PCs infected with the empty vector (EV), where the expression of TRAIL and GD2 tCAR is absent.

FIG. 3b shows the analysis of PC GD2 tCAR infected with the vector coding (which means that it carries the genetic information to produce a protein) the GD2 tCAR which is expressed in 79±7% of the cell population.

In FIG. 3c, the analysis of PC sTRAIL infected with a vector coding sTRAIL can be observed, where the presence of TRAIL has been confirmed in 99.3% of PC sTRAIL (28.3% on the cell membrane and 71% in the cytoplasm).

FIG. 3d shows the analysis of bi-functional PC sTRAIL (in the drawing sTRAIL Bi-Funct) producing both GD2 tCAR and also sTRAIL, where it is interesting to note that 74.2% of bi-functional PC sTRAIL express TRAIL (18.3% on the cell membrane and 55.9% in the cytoplasm) associated with an 85% expression of GD2 tCAR.

In FIG. 3e, the analysis of PC mTRAIL infected with the vector coding mTRAIL shows that TRAIL is produced by 95±8% of PC mTRAIL (55±7% on the cell membrane and 40±1% in the cytoplasm).

FIG. 3f shows the analysis of the bi-functional PC mTRAIL (in the drawing mTRAIL Bi-Funct) which produce both GD2 tCAR and mTRAIL.

In particular, TRAIL was detected in 71±4% of bi-functional PC mTRAIL (49±7% on the cell membrane and 22±3% in the cytoplasm) and 65±17% of these bi-functional PC mTRAIL are also positive for GD2 tCAR.

FIG. 4 is a graph showing the analysis of the release of sTRAIL by engineered PCs by means of ELISA (Enzime-Linked Immunosorbent Assay) evaluation.

The bi-functional PC sTRAIL were obtained by infecting once more the PC sTRAIL with a retroviral vector, in particular lentiviral, encoding GD2 tCAR.

The release of sTRAIL by bi-functional PC sTRAIL has been confirmed with an ELISA test obtaining an average of 253.5±10 pg/ml of sTRAIL.

PC EV and PC GD2 tCAR (the latter not present in the graph) do not spontaneously release TRAIL.

This evidence demonstrates that high levels of GD2 tCAR expressed by bi-functional PC sTRAIL do not significantly affect the production of sTRAIL, confirming the feasibility of the bi-functional targeting approach, according to the invention.

FIG. 5 shows the analysis of the expression of GD2 evaluated by FACS.

The cells were first selected according to the morphological parameters of FSC and SSC (left area) placing a gate on the cell population of interest and excluding debris, then the expression of GD2 was evaluated (right area, dark gray curve) by labeling with the primary murine antibody targeted against human GD2 (BD) and subsequent incubation with the secondary antibody conjugated to goat-produced fluorochrome APC and targeted against mouse antibodies (BD).

The secondary antibody has also been used alone to label cells as an isotypic control (right area, light gray line).

The levels of expression of GD2 are respectively high in line TC71 (FIG. 5a), low in line A673 (FIG. 5b) and absent in line RD-ED (FIG. 5c).

FIG. 6 shows the expression of both agonist receptors, that is, able to produce the physiological effect following the binding with the ligand (specifically DR4 and DR5) and decoy, therefore antagonists (specifically DcR1, DcR2) of TRAIL in ES cell lines (TC71 in black, A673 in dashed black and RD-ES in dotted black) by FACS analysis.

Figs. from 7a to 7f show the cytotoxicity exerted in vitro by bi-functional PC sTRAIL on ES target cell lines.

The PCs were labeled with the fluorescent molecule CFSE (Molecular Probes, Eugene, Oreg., USA), thus acquiring a green fluorescence.

More in detail, figs. from 7 to 7c show the cytotoxicity exerted by bi-functional PC sTRAIL with respect to ES cell lines evaluated with 24-hour co-culture assays, testing multiple target:effector (T:E) ratios, in detail T:E 1:1, 1:2 and 1:5. The cytotoxic effect of bi-functional PCs was further evaluated by comparing it with that induced by rhTRAIL (1 μg/ml) and by PCs expressing sTRAIL alone, while PC EV and PC GD2 tCAR were used as negative control.

Untreated control cells (referred to as CTR) of ES were introduced in the same way for comparison.

The green fluorescence of PCs labeled with CFSE allowed FACS to obtain a clear distinction between target cells (CFSE−) and effector cells (CFSE+).

The cytotoxic activity was evaluated by means of supravital labeling with propidium iodide (PI), placing a gate (that is, making a selection, gating) on the negative CFSE cells, in order to quantify only the percentage of dead cancer cells.

The reported p-values obtained with a Student's t or t-test statistical test and applied in all subsequent analysis, derive from multiple comparisons between PC sTRAIL and bi-functional PC sTRAIL with respect to control groups represented by PC EV, PC GD2 tCARs and by the treatment with rhTRAIL or by CTR tumor cells only.

For TC71 (FIG. 7a) we found: *p<0.01, °p<0.01, § p<0.01;

for A673 (FIG. 7b) we found: *p<0.05, °p<0.01, § p<0.01;

for RD-ES (FIG. 7 c) we found: *p<0.01, °p<0.01, § p<0.01.

In figs. from 7d to 7f, in order to evaluate the cytotoxicity of the sTRAIL molecule released by bi-functional PC sTRAIL, the tumor cells were seeded in a 12-well plate with a concentration of 6000 cells/cm² and the day after seeding they were incubated for 24 hours with the supernatant (that is, the growth medium conditioned by PCs) containing sTRAIL released in 48 hours by the PC sTRAIL and the bi-functional PC sTRAIL.

The mortality rate was evaluated using FACS by means of labelling with PI.

The supernatants deriving from PC EV and GD2 tCAR PC were used as controls. Control CTR ES cells were introduced in the same way for comparison.

The reported p-values concern multiple comparisons between PC sTRAIL and bi-functional PC sTRAIL with respect to control groups represented by PC EV, by PC GD2 tCAR and by the treatment with rhTRAIL or by CTR tumor cells only. For TC71 (FIG. 7d) we found: *p<0.05, °p<0.01, § p<0.05;

for A673 (FIG. 7e) we found: *p<0.01, °p<0.01, § p<0.01;

for RD-ES (FIG. 7f) we found: *p<0.01, °p<0.01, § p<0.01.

FIGS. 8a and 8b show the anti-tumor activity of bi-functional PC sTRAIL in a three-dimensional (3D) model with respect to spheroids of TC71 tumor cells.

The TC71 cell line of ES was grown in tumor spheroids “in vivo-like”, that is, mimicking the tumor in vivo, in order to more realistically study the infiltration and cytotoxicity of bi-functional PC sTRAIL in a 3D model in vitro.

The formation of tumor spheroids was obtained by seeding 20,000 TC71 cells per well of a multi-well plate with 96 wells with a U-shaped bottom and ultra-low adherence (Corning, N.Y., USA), characteristics that allow over time cell-to-cell aggregation and therefore the formation of the spheroid.

The PCs were added 24 hours after the seeding of TC71 at a concentration of 25,000 cells per well.

A 15-hour co-culture period was identified as optimal to quantify the cytotoxicity of bi-functional PC sTRAIL, by means of luminescence-based assays, in detail the Cell Titer-Glo® 3D Cell Viability assay (Promega, Madison, Wis., USA), which measures the ATP content present within the spheroid, as an indicator of cell vitality, and the Caspase-Glo® 8 (Promega) assay that measures caspase 8 activity.

Analyzing cell vitality (FIG. 8a), it can be noted that the ATP content is lower in co-cultures with bi-functional PC sTRAIL and with PC sTRAIL, compared to control co-cultures with PC EV and PC GD2 tCAR.

In detail, we found: *p<0.001, °p<0.01, § p<0.01.

In parallel, it can be observed that bi-functional PC sTRAIL are able to strongly activate caspase 8 in TC71 spheroids (FIG. 8b), compared to non-cytotoxic control PCs (that is, PC EV and PC GD2 tCAR) and in a similar measure to treatment with rhTRAIL or to co-culture with PC sTRAIL.

Specifically, we found: *p<0.01, °p<0.001, § p<0.001.

FIG. 9 comprises three graphs showing the result of cell-to-cell interaction assays where it is evident how GD2 tCAR expressed on the surface of bi-functional PC sTRAIL increases the binding with ES cell lines expressing GD2.

The PCs labeled with CFSE were seeded in a 12-well plate to create a monolayer of confluent cells in 12 hours.

In order to test cell-to-cell interaction, a suspension of ES cells expressing DsRed was added over the PC monolayer.

After one hour and a half of incubation, the medium was removed and the co-cultures were washed twice with PBS (phosphate-buffered saline), then stirred for 3 minutes in order to dissociate the weakly adhering tumor cells and the medium was removed again.

The remaining cells were harvested by means of trypsinization and re-suspended in a fixed volume of PBS.

The absolute number of aggregates between PCs and ES cells was evaluated using FACS quantifying the doubly positive CFSE/DsRed population in a constant fraction of time of 60 seconds, as better described below in Example 1.

The datum was expressed as “fold-change”, that is, as the ratio between the number of cell aggregates deriving from the interaction of the engineered PCs (PC GD2 tCAR, PC sTRAIL and bi-functional PC sTRAIL) with ES cells and the number of aggregates produced by the interaction of PC EV with ES cells.

This was done for all three ES lines considered.

The PCs engineered with GD2 tCAR, both PC GD2 tCAR and also bi-functional PC sTRAIL, bind the GD2 positive, TC71 and A673 cell lines, more than PC EV and PC sTRAIL (for the TC71 *p<0.01, °p<0.01; for the A673 *p<0.05, °p<0.01).

No differences were found between the different types of PCs in the capacity to form cell aggregates with the RD-ES line which is negative for GD2.

### DETAILED DESCRIPTION OF EXAMPLES OF PREFERRED EMBODIMENTS

In the following examples, for simplicity reference has been made to human-derived cells and/or pericytes, however the person of skill understands that the use of animal-derived cells and/or pericytes is also possible.

### Example 1

Role Played by GD2 tCAR on the Membrane of Bi-Functional PCs in the Interaction Between Bifunctional PC mTRAIL and ES Cells.

The GD2 tCAR technology was further tested by considering PCs producing a form of TRAIL bound to the membrane (mTRAIL), as in FIG. 1b (indicated with 3′), which requires direct cell-to-cell contact in order to induce apoptosis of the cancer cell.

Thanks to the new GD2 tCAR receptor, mTRAIL-producing bi-functional PCs were specifically directed against GD2-expressing ES and the cell-to-cell interaction was optimized.

The selective recognition of the cell target by the PCs engineered with GD2 tCAR was evaluated with respect to all ES cell lines, which differ considerably in the levels of expression of GD2, which are high in TC71, low in A673 and completely absent in RD-ES, as negative control cell line.

A monocistronic retroviral vector based on the murine stem cell virus (MSCV) and coding a red fluorescent protein called DsRed was used to stably infect the ES cell lines, so as to distinguish the latter from the PCs in the interaction assay.

The retrovirus was produced by means of the FLYRD18 cell packaging line (which allows to produce a virus that is infectious but unable to replicate itself). The PCs were labeled with the CFSE fluorescent dye as per the reagent protocol. The cell-to-cell interaction assay was performed as described below.

The PCs labeled with CFSE were seeded in a 12-well high-density plate (100,000 cells/well) to create a monolayer of confluent cells in 12 hours.

In order to test the interaction, a suspension of ES cells expressing DsRed (70,000 cells/well) were added to the PC monolayer.

After an hour and a half of incubation, the medium was removed, and the co-cultures were washed twice with PBS and stirred for 3 minutes in order to dissociate the tumor cells weakly bound to the monolayer.

The remaining cells were harvested by means of trypsinization and resuspended in a fixed volume of PBS.

The absolute number of the PC-ES cell aggregates was quantified using FACS considering the double positive population CFSE/DsRed, in a constant fraction of time of 60 seconds.

The datum was expressed as “fold-change” between the number of cell aggregates deriving from the interaction of the different engineered PCs (PC GD2 tCAR, PC mTRAIL and bi-functional PC mTRAIL) with ES cells and the number of aggregates produced by the interaction of PC EV with ES cells.

The binding stability in the population of aggregates was also evaluated comparing PC EV and PC GD2 tCAR in the interaction with the TC71 line of ES.

After the dissociation with trypsin, the PC-TC71 cell aggregates were maintained in interaction for further respective periods of 2 and 4 hours under stringent conditions, that is, such as to hinder the maintenance of less firm bindings, specifically at 4° C. on a rotating support.

The absolute number of aggregates was then quantified using FACS.

The data were expressed by comparing the absolute number of aggregates at 2 or 4 hours in stringent conditions with the absolute number after dissociation with trypsin.

RESULTS of example 1.

According to the invention, the engineering of the surface of PCs with GD2 tCAR represents a method able to strengthen the interaction between PCs and ES cells expressing GD2, also forcing the binding of mTRAIL to its agonist receptors on ES cells.

In order to study the new properties provided by GD2 tCAR, the ability of bi-functional PC mTRAIL to bind ES cells expressing GD2 was tested in a cell-to-cell interaction assay.

For this purpose, the PCs were labeled with the CFSE molecule, thus acquiring a green fluorescence; on the contrary, the ES cell lines were stably infected with a viral vector coding the DsRed gene, showing a red fluorescence.

Thanks to this different labelling, the PCs and the ES cells proved to be clearly distinguishable using FACS and their aggregates appeared as double positives for both fluorescent labels, CFSE and DsRed (FIG. 10a, gate P4).

In the cell-to-cell interaction test, ES DsRed cells were added to the PC monolayer labelled with CFSE left in interaction for 1.5 hours.

After sequential washings to lose weak interactors, the cells were harvested and analyzed using FACS in order to determine the absolute number of PC-ES cell aggregates, displayed as double positive CFSE/DsRed events.

The corresponding gating strategy has been described in FIG. 10a.

The cells were first selected according to the FSC and SSC morphological parameters (FIG. 10a, left area) in order to exclude the debris, then the DsRed-ES cells and the CFSE-PC were displayed in a CFSE and DsRed dot plot (that is, a dot dispersion diagram representing the correlation between two parameters) in order to obtain the double positive events, corresponding to the PC-ES cell aggregates, displayed in the gate on the upper right (P4; FIG. 10a, middle area). A gate of the double positive population CFSE/DsRed in P1 (FIG. 10a, right area) confirmed the larger sizes and greater internal cell complexity of the PC-ES aggregates.

The number of aggregates produced by the interaction between the PC GD2 tCAR, the bi-functional PC mTRAIL and the ES cells, was compared to the number of aggregates between the PC EV and ES cells and the data shown as fold-change, for all ES cell lines tested (FIGS. 10b and 10c).

As expected, the values expressed by the ratios are directly proportional to the abundance of GD2 in the ES cell lines and to the ability of the PCs to interact through functionalization with GD2 tCAR.

The PCs engineered with GD2 tCAR, both PC GD2 tCAR and also bi-functional PC mTRAIL, bind the highly GD2 positive TC71 line at least twelve times better than the PC EV and PC mTRAIL (FIG. 10b, left area). In particular, we found: *p<0.001, °p<0.001.

Furthermore, by incubating the aggregates for 2 hours in rotation at 4° C. (FIG. 10b, right area), the binding of PC GD2 tCAR to TC71 is more stable than that of PC EV.

The binding capacity of PC GD2 tCAR did not decrease significantly compared to the base value at the time of dissociation, on the contrary, that of PC EV is lower (p<0.001).

After 4 hours in stringent conditions, the number of aggregates of PC GD2 tCAR is still stable compared to the base condition while that of PC EV has further decreased if compared to both the time-point (that is, at the time of detection) of 2 hours under stringent conditions and also to the base disassociation time (*p<0.05).

Also comparing the two incubation times of 2 and 4 hours in stringent conditions with each other, there are no differences in the number of aggregates with PC GD2 tCAR, while there was a decrease in the number of aggregates for PC EV at 4 hours.

A significant but more reduced gain in terms of the binding capacity of PCs engineered with the GD2 tCAR, both PC GD2 tCAR and also bi-functional PC mTRAILs, was found with the A673 line expressing low levels of GD2 (FIG. 10c, area on the left; we found: *p<0.05, °p<0.05).

With regards to the negative RD-ES GD2 line, we found no difference in terms of formation of PC-ES aggregates among the different types of engineered PCs, since this tumor cell line does not have the GD2 ligand on the surface (FIG. 10c, right area).

The PCs engineered with GD2 tCAR have therefore demonstrated an optimized and specific binding for ES cells expressing the GD2 antigen.

### Example 2

Evaluation of the Cytotoxic Effect of Bi-Functional PC mTRAIL on an ES Cell Line.

According to the invention, it has been demonstrated that bi-functional PCs, engineered to produce both mTRAIL and also GD2 tCAR simultaneously, maintain TRAIL-mediated cytotoxic activity also improving the interaction with GD2 positive tumor cells.

First, two-dimensional (2D) co-cultures were produced as follows. The PCs were labeled with the CFSE label and seeded in a 12-well culture plate. 12 hours after seeding, the tumor cells were added at a density of 6,000 cells/cm². Different T:E ratios (1:1, 1:2, and 1:5) were tested.

The anti-tumor activity of PCs expressing mTRAIL was evaluated using FACS by means of labeling with PI (50 μg/ml) after only 12 hours of co-culture, selecting the CFSE negative and PI positive cells, that is, the dead tumor cells. rhTRAIL at a dose of 1 μg/ml was introduced as positive control.

Subsequently, the anti-tumor activity was confirmed by caspase 8 activation assay.

In order to demonstrate that bi-functional PC mTRAIL are able to send ES cells into apoptosis by activating the TRAIL pathway (that is, the signal transduction pathway), the activation pattern of caspase 8 was analyzed, which is an early marker of TRAIL-mediated apoptosis, by means of western blot technique on a lysate of TC71 cells, after 4 hours of co-culture.

In detail, the engineered PCs were seeded at a density of 30,000 cells/cm² in a 12-well plate.

After 12 hours, TC71s were added at the density of 6,000 cells/cm², therefore at a T:E ratio of 1:5.

rhTRAIL was introduced at a dose of 1 μg/ml as positive control.

The cells were harvested and lysed after 4 hours of co-culture with the engineered PCs or of incubation with rhTRAIL.

The protein lysate was quantified using the Biorad Protein Assay (Bio-Rad, Hercules, Calif., USA).

According to western blot, after the transfer of proteins from the gel to the membrane, two measurements were carried out using antibodies: the first to detect caspase 8 using a murine monoclonal antibody (mAb) (1C12; Cell Signaling Technologies, Beverly, Mass., USA) as primary antibody and a secondary IRDye 800CW antibody (LI-COR Biosciences, Lincoln, Nebr., USA) goat-produced and targeted against the mouse antibodies.

In the second, GAPDH was detected using a rabbit mAb (14C10; Cell Signaling Technologies) as primary antibody and a secondary IRDye 800CW antibody (LI-COR Biosciences) goat-produced and targeted against the rabbit antibody.

The cytotoxic potential of bi-functional PC mTRAIL was further confirmed in a 3D model: the tumor spheroids are heterogeneous cell aggregates which, when their diameter becomes larger than 500 μm, are characterized by the presence of hypoxic zones and a necrotic center.

The 3D spheroids are therefore considered valid models for modeling the typical characteristics of tumor micro-regions or micro-metastases.

In order to create these tumor spheroids, 96-well plates were used, with a U-shaped bottom and ultra-low adherence (Corning), which, in contrast to the standard methods, do not require a coating of the surface to prevent cell adherence.

The tumor cells expressing DsRed formed a 3D structure in these plates in 24 hours.

The U shape of the well promotes the formation of individual spheroids with reproducible sizes and located in the center of the well.

After 24 hours, the engineered PCs labeled with the cell tracer CFSE were added to the tumor spheroid, at the density of 25,000 cells/well.

rhTRAIL was again introduced at a dose of 1 μg/ml as positive control.

The architecture of the spheroids was monitored using fluorescence microscope for 48 hours of co-culture, in order to follow the PC infiltration and the cytotoxic effect mediated by bi-functional PC mTRAIL on the tumor spheroid.

Furthermore, the vitality of the cells that form the spheroid was quantified after 15 hours of co-culture with luminescence-based assays, in this specific case the CellTiter-Glo® 3D cell Viability Assay (Promega), which measures the ATP content present inside the spheroid, as an indicator of cell vitality, and the Caspase-Glo® 8 (Promega) which measures the activity of caspase 8.

RESULTS of example 2.

Cytotoxicity assays were introduced to verify that the simultaneous expression of the pro-apoptotic ligand TRAIL and of the GD2 tCAR molecule on the surface of the PCs does not invalidate the cytotoxic potential exerted by the engineered PCs by means of cell-to-cell contact with tumor cells.

First, the cytotoxicity of bi-functional PC mTRAIL was confirmed with a co-culture experiment with the ES TC71 line, testing different time-points, precisely at 12 hours (FIG. 11a) 24 and 48 hours (data not shown) and multiple T:E ratios (1:1; 1:2 and 1:5).

The cytotoxic effect of bi-functional PC mTRAIL was compared with that induced by rhTRAIL (1 μg/ml) and by PCs expressing mTRAIL only, while PC EV and PC GD2 tCAR were used as negative controls.

The bi-functional PC mTRAIL were able to exert a significant cytotoxic effect, with the highest mortality level (up to 80±8% of dead cells detected with the T:E ratio of 1:5) reached after only 12 hours of co-culture, comparable to that produced by PC mTRAIL and by the treatment with rhTRAIL.

The cytotoxicity mediated by bi-functional PC mTRAIL also increases proportionally with the increase of the T:E ratio (p<0.05).

For all the time-points evaluated, the co-culture of TC71 with PC EV or PC GD2 tCAR does not alter the vitality of the tumor cells. From the statistical analysis using t-test we found: *p<0.05; °p<0.001; °p<0.001, § p<0.001.

In order to demonstrate that bi-functional PC mTRAIL are able to send sarcoma cells into apoptosis by activating the TRAIL pathway, the activation pattern of caspase 8 was analyzed with western blot on a lysate of TC71 cells after only 4 hours of co-culture (FIG. 11b).

The TC71 cells were co-cultured with engineered PCs at the T:E ratio of 1:5 or in the presence of rhTRAIL at a dose of 1 μg/ml, as positive control.

For the TC71 in co-culture both with bi-functional PC mTRAIL and also with PC mTRAIL or treated with rhTRAIL we observed the presence of a band corresponding to the p43/p41 fragment of caspase 8, resulting from the activation and the consequent cleavage of the latter.

In co-culture with the control PCs, PC EV and PC GD2 tCAR, no activation of caspase 8 was found, similarly to the control represented by TC71 from only CTR.

In conclusion, these data confirmed that bi-functional PC mTRAIL induce apoptosis following the same pathway of rhTRAIL.

In vivo-like spheroids were also obtained starting from the TC71 cell line expressing DsRed to better analyze the infiltration and cytotoxicity of bi-functional PC mTRAIL labeled with CFSE in a 3D model.

Therefore, the ability of the TC71 line to form tumor spheroids was tested (FIG. 12a, first line).

The formation of tumor spheroids was obtained by seeding TC71 in a 96-well plate, with a U-shaped bottom and ultra-low adherence (Corning).

The formation of the spheroids is a bi-phase process of cell aggregation and spheroid maturation that leads to a compaction and a significant reduction in volume, reaching a size of 500-600 μm after 24 hours from seeding.

The spheroids of TC71 remain stable for several days with minimal morphological alterations and maintain high levels of expression of the GD2 antigen (data not shown).

Subsequently, the spheroids of TC71 were treated with rhTRAIL (1 μg/ml) in order to evaluate whether the 3D conformation confers upon them resistance to treatment with the death ligand. As can be seen in FIG. 12a, second line, rhTRAIL triggers the apoptosis of TC71, breaking the 3D architecture and reducing both the fluorescence and the size of the spheroids.

The tumor spheroids were first monitored with a fluorescence microscope in order to observe the infiltration and cytotoxicity of the engineered PCs during the 48 hours of co-culture (FIG. 12b).

In the first 8 hours of co-culture, all the different types of engineered PCs are progressively localized on the periphery of the tumor spheroid (FIG. 12b, first column) and then begin to interact with the tumor cells at 24 hours, causing a compaction of the red spheroid (FIG. 12b, second column).

Differences between the different engineered PCs appeared after 48 hours of co-culture (FIG. 12b, third and fourth column).

Both PC EV and also PC GD2 tCAR have deeply infiltrated the red spheroid, reaching its center, as evidenced by the analysis of the frozen sections (FIG. 12b, last column), while both PC mTRAIL and also bi-functional PC mTRAIL have progressively destroyed the red spheroid starting from the edges with a consequent decrease in the fluorescence of the DsRed expressed by the tumor cells and the complete loss of the spheroidal architecture, as observed in the frozen sections.

An early 15-hour time point was then chosen to quantify the cytotoxicity of bi-functional PC mTRAIL using luminescence-based assays (FIG. 12c).

The cytotoxicity of bi-functional PC mTRAIL was evidenced both in terms of decreased cell vitality and also of activation of caspase 8 in the tumor spheroid. The ATP content (FIG. 12c, left and middle area), as an index of cell vitality, is lower in co-cultures with bi-functional PC mTRAIL and with PC mTRAIL if compared with co-cultures with control PCs, PC EV and PC GD2 tCAR. In particular, we found: *p<0.001, °p<0.001, § p<0.001 In parallel, bi-functional PC mTRAIL are able to activate caspase 8 in TC71 (FIG. 12c, right area) to a similar extent to PC mTRAIL and the treatment with rhTRAIL, while PC EV and PC GD2 tCAR do not activate the apoptotic pathway. From the statistical analysis we obtain: *p<0.05, °p<0.001, § p<0.001.

Overall, these in-vitro data have shown that bi-functional PC mTRAIL preserve a significant anti-tumor activity, comparable to that of PC mTRAIL, despite the simultaneous expression of high levels of GD2 tCAR.

Even more relevant is that the anti-tumor action of bifunctional PC mTRAIL has been further strengthened by the increased targeting capacity due to the presence of GD2 tCAR which has the function of improving interactions with tumor cells expressing GD2, as presented in example 1.

### Example 3

Effect of PC Engineering with GD2 tCAR in the Interaction Between PCs and GBM.

As a first step, in a manner similar to what was described in example 1, the binding affinity of bi-functional PC mTRAIL with regards to the T98G line of GBM, which is characterized by a high expression of GD2, was evaluated with a cell-to-cell interaction assay.

The cell-to-cell interaction assay was then further set up to evaluate whether the optimized binding capacity of bi-functional PC mTRAIL with regards to GD2-positive GBM cells translates into a more effective cytotoxicity, compared to that mediated by PC mTRAIL.

For this purpose, the T98G line of GBM, highly GD2-positive, was considered, and the interaction assay was conducted for a period of 7 hours.

Subsequently, a labelling was performed with Annexin V, an apoptosis marker, on the PC-GBM cell aggregate population produced in the interaction assay. The CFSE/DsRed aggregates positive for Annexin V were then quantified using FACS, in a constant fraction of time of 60 seconds.

Data were expressed as a percentage of PC-GBM cell aggregates positive for Annexin V.

Anti-idiotype sera produced in mice and targeted against GD2 tCAR were then used to block the GD2 tCAR-mediated interaction between bi-functional PC mTRAIL and T98G and the effect on the apoptosis was then analyzed using labeling with Annexin V.

In brief, the engineered PCs were incubated with these sera diluted 1:20 for 40 minutes at 37° C. and 5% CO2 and then used to set up a cell-to-cell interaction assay, as described previously.

The percentage of PC-GBM cell aggregates positive for Annexin V after 7 hours of interaction was then analyzed using FACS.

RESULTS of example 3.

According to the invention, PCs engineered with GD2 tCAR, both PC GD2 tCAR and bi-functional PC mTRAIL, bind the T98G line resulting as highly positive for GD2 expression (as indicated by analysis using FACS in FIG. 13) at least four times more than PC EV and PC mTRAIL (FIG. 14); *°p<0.001).

It is interesting to note that the co-expression of mTRAIL and GD2 tCAR confers upon bi-functional PC mTRAIL a much faster cytotoxic activity against GD2-positive GBM cells, as will be better described below.

Cell mortality was evaluated at an early time-point of 7 hours, using labelling with Annexin V on the double positive aggregates CFSE/DsRed.

The gating strategy previously described for the interaction assay (with reference to FIG. 10a) was applied in this experiment and the percentage of PC-GBM aggregates positive for Annexin V was quantified on the cell population comprised in the gate P4 of FIG. 15a.

The interaction with bi-functional PC mTRAIL produced a significant cytotoxic effect on the aggregates (59±5% of apoptotic cells), if compared with both PC mTRAIL (32±7%) and also control PCs, that is, PC EV (9±3%) and PC GD2 tCAR (154%) (p<0.001) (FIG. 15b, columns without dashed lines).

In order to confirm the role of GD2 tCAR expressed by bi-functional PC mTRAIL in inducing a more rapid and effective apoptosis, anti-idiotype mouse sera were used against the chimeric receptor to block the binding between GD2 tCAR and the GD2 ligand (FIG. 15b, columns with dashed lines).

By blocking the interaction between GD2 tCAR and GD2 by means of incubation of bi-functional PC mTRAIL with mouse sera, the percentage of aggregates positive for Annexin V was significantly reduced up to 40±2%, comparable to PC mTRAIL with (36±7%) or without incubation with sera. Applying the statistical analysis according to t-test we found: *p<0.001, **p<0.001, °p<0.001, ° °p<0.001.

These data suggest that the expression of GD2 tCAR by bi-functional PC mTRAIL confers a specific targeting of tumor cells expressing GD2 and also enhances its cytotoxic effect.

It has been found that the invention achieves the intended purposes.

The invention as conceived is susceptible to modifications and variants, all of which come within the scope of the inventive concept.

In practice, the elements indicated can be replaced with other technically equivalent ones, without departing from the field of protection of the following claims.

1. A method for the production of bi-functional cells, comprising engineering starting cells obtaining engineered cells contemporarily expressing both a molecule having an anti-tumor activity and a fraction of a monoclonal antibody directed against a tumor antigen.

2. The method as in claim 1, wherein said engineering comprises:
inserting into said starting cells a coding sequence for a pro-apoptotic molecule; and
inserting into said starting cells a coding sequence for a fraction of a monoclonal antibody directed against a tumor antigen.

3. The method as in claim 2, wherein said pro-apoptotic molecule is TRAIL.

4. The method as in claim 3, wherein said TRAIL molecule released by said engineered cells comprises a molecule of soluble TRAIL, or sTRAIL, which is equipped with a secretion sequence.

5. The method as in claim 3, wherein said TRAIL molecule released by said engineered cells comprises a molecule of membrane-bound TRAIL, or mTRAIL, which is equipped with a sequence of binding to the membrane of said engineered cells.

6. The method as in claim 1, wherein said fraction of a monoclonal antibody is a scFv taken from an immunoglobulin directed toward the tumor antigen.

7. The method as in claim 6, wherein said scFv is comprised in a chimeric antigen receptor, known as CAR.

8. The method as in claim 7, wherein said CAR is truncated CAR, or tCAR, of the intracell signal transduction domains.

9. The method as in claim 8, wherein said tumor antigen is GD2.

10. The method as in claim 9, wherein said tCAR has binding affinity for the GD2 antigen.

11. The method as in claim 1, wherein said engineering comprises infecting said starting cells with at least one viral vector.

12. The method as in claim 11, wherein said viral vectors comprise retrovirus.

13. The method as in claim 12, wherein said retrovirus comprises lentivirus.

14. The method as in claim 1, wherein said starting cells are human pericytes, or PC.

15. The method as in claim 14, wherein said starting cells have origin chosen among adipose tissue, osteo-medullary tissue, placenta, amniotic fluid, dental pulp, muscle tissue, cardiac tissue, umbilical cord, cutaneous tissue, pancreatic tissue, intestinal tissue, decidual endometrial tissue.

16. The method as in claim 15, wherein said starting cells are cells of the adipose tissue, or AD-PC.

17. The method as in claim 16, further comprising permanently modifying said AD-PC.

18. The method as in claim 1, wherein said starting cells are chosen among: human cells of autologous or allogeneic origin, animal cells.

19. The method as in claim 1, wherein said bi-functional cells comprise:
a nucleus and a cytoplasm;
a permanent modification of said nucleus, wherein said permanent modification comprises a TRAIL molecule and a GD2 tCAR molecule codified by at least one viral vector and inserted in said nucleus of said starting cells.

20. The method as in claim 1, wherein said tumor antigen is GD2.


SEQUENCE LISTING (9 sequences)

SEQ ID NO: 1 - DNA, 8242 bp, Unknown
  GD2 tCAR expression vector
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgataattcgataattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgcggccgaggcaggccacgcgtcccgggacgcgtgcggccgcggccgagcaggccggatccatgggctggagcctgatcctcctgttcctcgtcgctgtggctacaggtaaggggctcacagtagcaggcttgaggtctggacatatatatgggtgacaatgacatccactttgcctttctctccacaggtgtgcactctgatattgtgatgacccagtctccatcctccctggctgtgtcagcaggagagaaggtcactatgaactgcagatccagtcagagtctcctcagcagtagaacccgaaagaactacttggcttggtaccagcagaaaccagggcagtctcctaaactgctgatctactgggcatctattagggaatctggggtccctgatcgcttcacaggcagtggatctgggacagatttcactctcaccatcagcagtgtgcaggctgaagacctggcagtttattactgcaagcaatcttataatcttcggacgttcggtggaggcaccaagctggaaatcaaaggcagcactagtggtagcggcaaaccaggttccggcgaaggctcgagcaaaggtgaggtgcagctgcaggagtctggacctggcctggtggcgccctcacagagcctgtccatcacttgcactgtctctgggttttcattacccagctatggtgttcactgggttcgccagcctccaggaaagggtctggagtggctgggagtaatctgggctggtggaatcacaaattataactcggctctcatgtccagactgaccatcagcaaagacaactccaagagccaagttttcttaaaaatgaacagtcttcaaactgatgacacagccatatactactgtgccagaggcggctctgattacgacggctttgcttactggggccaagggactctggtcactgtctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 2 - DNA, 1090 bp, Unknown
  nucleotidic sequence of GD2 tCAR
atgggctggagcctgatcctcctgttcctcgtcgctgtggctacaggtaaggggctcacagtagcaggcttgaggtctggacatatatatgggtgacaatgacatccactttgcctttctctccacaggtgtgcactctgatattgtgatgacccagtctccatcctccctggctgtgtcagcaggagagaaggtcactatgaactgcagatccagtcagagtctcctcagcagtagaacccgaaagaactacttggcttggtaccagcagaaaccagggcagtctcctaaactgctgatctactgggcatctattagggaatctggggtccctgatcgcttcacaggcagtggatctgggacagatttcactctcaccatcagcagtgtgcaggctgaagacctggcagtttattactgcaagcaatcttataatcttcggacgttcggtggaggcaccaagctggaaatcaaaggcagcactagtggtagcggcaaaccaggttccggcgaaggctcgagcaaaggtgaggtgcagctgcaggagtctggacctggcctggtggcgccctcacagagcctgtccatcacttgcactgtctctgggttttcattacccagctatggtgttcactgggttcgccagcctccaggaaagggtctggagtggctgggagtaatctgggctggtggaatcacaaattataactcggctctcatgtccagactgaccatcagcaaagacaactccaagagccaagttttcttaaaaatgaacagtcttcaaactgatgacacagccatatactactgtgccagaggcggctctgattacgacggctttgcttactggggccaagggactctggtcactgtctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctaa

SEQ ID NO: 3 - protein, 335 aa, Unknown
  protein sequence of GD2 tCAR
MGWSLILLFLVAVATGVHSDIVMTQSPSSLAVSAGEKVTMNCRSSQSLLSSRTRKNYLAWYQQKPGQSPKLLIYWASIRESGVPDRFTGSGSGTDFTLTISSVQAEDLAVYYCKQSYNLRTFGGGTKLEIKGSTSGSGKPGSGEGSSKGEVQLQESGPGLVAPSQSLSITCTVSGFSLPSYGVHWVRQPPGKGLEWLGVIWAGGITNYNSALMSRLTISKDNSKSQVFLKMNSLQTDDTAIYYCARGGSDYDGFAYWGQGTLVTVSTTTPAPRPPTPAPTIASQPLSLRPEACRPAAGGAVHTRGLDFACDIYIWAPLAGTCGVLLLSLVITLYC

SEQ ID NO: 4 - DNA, 7894 bp, Unknown
  sTRAIL expression vector
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgataattcgataattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgcggccgaggcaggccacgcgtcccgggatgggctggagcctgatcctcctgttcctcgtcgctgtggctacaggtaaggggctcacagtagcaggcttgaggtctggacatatatatgggtgacaatgacatccactttgcctttctctccacaggtgtgcactcgggatccagtgctcggaatcgacagaagcgacggatgaagcagatcgaggacaagattgaggaaatcctgtccaagatttaccacatcgagaacgagatcgctcgaatcaagaagctcattggagagagggtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 5 - DNA, 775 bp, Unknown
  nucleotidic sequence of sTRAIL
atgggctggagcctgatcctcctgttcctcgtcgctgtggctacaggtaaggggctcacagtagcaggcttgaggtctggacatatatatgggtgacaatgacatccactttgcctttctctccacaggtgtgcactcgggatccagtgctcggaatcgacagaagcgacggatgaagcagatcgaggacaagattgaggaaatcctgtccaagatttaccacatcgagaacgagatcgctcgaatcaagaagctcattggagagagggtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 6 - protein, 230 aa, Unknown
  protein sequence of sTRAIL
MGWSLILLFLVAVATGVHSGSSARNRQKRRMKQIEDKIEEILSKIYHIENEIARIKKLIGERVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 7 - DNA, 7965 bp, Unknown
  mTRAIL expression vector
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgataattcgataattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgcggccgaggcaggccacgcgtcccgggatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 8 - DNA, 846 bp, Unknown
  nucleotidic sequence of mTRAIL
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 9 - protein, 281 aa, Unknown [sequence omitted]